(19) United States
(12) Patent Application Publication
(10) Pub. No.: US 2017/0042981 A1
(43) Pub. Date: Feb. 16, 2017

(54) HALF-LIFE EXTENDED FACTOR FVIIA PROTEIN FOR PREVENTION AND TREATMENT OF BLEEDING AND DOSING REGIMENS THEREFOR

(71) Applicant: CSL LIMITED, Parkville (Victoria) (AU)

(72) Inventors: Debra BENSEN-KENNEDY, Phoenixville, PA (US); Alex VELDMAN, Kronberg/taunus (DE); Sabine ZOLLNER, Muri (CH); Eva HERZOG, Gladenbach (DE)

(73) Assignee: CSL LIMITED, Parkville (Victoria) (AU)

(21) Appl. No.: 15/302,597

(22) PCT Filed: Mar. 30, 2015
(86) PCT No.: PCT/AU2015/050137

§ 371 Date: Oct. 7, 2016

Related U.S. Application Data
(60) Provisional application No. 61/978,218, filed on Apr. 11, 2014.

(30) Foreign Application Priority Data
May 9, 2014 (EP) 14167612.2
May 15, 2014 (EP) 14168389.6

(51) Int. Cl. A61K 38/48; A61K 38/38; A61K 9/00; A61K 47/48

(52) U.S. Cl. CPC A61K 38/4846; C12Y 304/21021; A61K 47/48215; A61K 38/38; A61K 9/0019

(57) ABSTRACT

The present invention relates to dosing regimens with half-life extended Factor VIIa (FVIIa) for prophylactic and “on-demand” treatment of bleeding, as well as for preventing a bleeding episode during or after surgery in patients with congenital or acquired bleeding disorders. The present invention further relates to the use of half-life extended FVIIa for treating or preventing blood loss in patients without bleeding disorders in situations of hemorrhage, i.e., due to trauma or surgery. Another aspect of the invention is the treatment of acquired haemophilia.

### RELATED APPLICATION DATA

The present application claims priority from U.S. Patent Application No. 61/978218 filed 11 Apr. 2014, from European Patent Application No. 14167612.2 filed 9 May 2014 and from European Patent Application No. 14168389.6 filed 15 May 2014. The entire contents of all applications are hereby incorporated by reference.

### SEQUENCE LISTING

The present application is filed with a Sequence Listing in electronic form. The entire contents of the Sequence Listing are hereby incorporated by reference.

### FIELD OF THE INVENTION

The present invention relates to dosing regimens with half-life extended Factor VIIa (FVIIa) for prophylactic and “on-demand” treatment of bleeding, as well as for preventing a bleeding episode during or after surgery in patients with congenital or acquired bleeding disorders. The present invention further relates to the use of half-life extended FVIIa for treating or preventing blood loss in patients without bleeding disorders in situations of hemorrhage i.e., due to trauma or surgery. Another aspect of the invention is the treatment of acquired haemophilia.

### BACKGROUND OF THE INVENTION

Hemophilia A is an inherited coagulation disorder. It results from a chromosome X-linked deficiency of blood coagulation Factor VIII, and affects almost exclusively males with an incidence between one and two individuals per 10,000. The X-chromosome defect is transmitted by female carriers who are not themselves clinically symptomatic. The clinical manifestation of hemophilia A is an increased bleeding tendency. Before replacement therapy with Factor VIII concentrates was introduced, the mean life span for a person with severe hemophilia was less than 20 years. The use of concentrates of Factor VIII generated from plasma and later on of recombinant forms of Factor VIII has considerably improved the situation for hemophilia patients, increasing the mean life span extensively and giving most of them the possibility to live a more or less normal life. Hemophilia B being 5 times less prevalent than hemophilia A is caused by non-functional or missing Factor IX and is treated with Factor IX concentrates from plasma or a recombinant form of Factor IX.

The goal of therapy for hemophilia is to treat or prevent hemorrhage, thereby reducing disabling joint and tissue damage, and improving quality of life (QoL). In both hemophilia A and in hemophilia B, the most serious medical problem in treating the disease is the generation of inhibitory alloantibodies against the replacement factors. Up to 30% of all hemophilia A patients develop inhibitory antibodies to Factor VIII. Inhibitory antibodies to Factor IX occur to a lesser extent but with more severe consequences, as they are less susceptible to immune tolerance induction therapy and have a higher potential to trigger allergic reactions when binding to FIX. The treatment for patients with hemophilia A (FVIII deficiency) or hemophilia B (FIX deficiency) who have developed inhibitory antibodies (Congenital Hemophilia with Inhibitors, CHwI) to FVIII or FIX (especially high titer inhibitors) is challenging, since normal replacement with Factor VIII or IX is not effective.

Another immune reaction to Factor VIII or Factor IX leading to the formation of autoantibodies inhibiting Factor VIII or FIX activity or function is referred to as acquired hemophilia A or acquired hemophilia B, respectively. This condition usually occurs in patients not having any deficiencies in Factor VIII or in Factor IX. Acquired hemophilia is a rare condition, with a yearly incidence of 0.2-1.0 per million population. The majority of cases are due to autoantibodies to Factor VIII, only few cases of acquired hemophilia B are reported. The autoantibodies are mainly IgG4 antibodies which bind to the coagulation factors and partly or completely neutralize their activation or function or accelerate their clearance. This results in life-threatening hemorrhage in a high proportion of affected patients. Common sites of bleeding are skin, mucosa, muscles and retroperitoneum, in contrast to patients with hereditary hemophilia who bleed predominantly into joints and muscles. Similar to CHwI, acquired hemophilia can be treated with an activated prothrombin complex concentrate or recombinant activated Factor VII (NovoSeven®, Novo Nordisk) to control bleeding episodes.

The current model of coagulation states that the physiological trigger of coagulation is the formation of a complex between tissue Factor (TF) and Factor VIIa (FVIIa) on the surface of TF expressing cells, which are normally located outside the vasculature. This leads to the activation of Factor IX and Factor X ultimately generating some thrombin. In a positive feedback loop thrombin activates Factor VIII and Factor IX, the so-called “intrinsic” arm of the blood coagulation cascade, thus amplifying the generation of Factor Xa, which is necessary for the generation of the full thrombin burst to achieve complete hemostasis. It was shown that by administering supraphysiological concentrations of Factor VIIa hemostasis is achieved bypassing the need for Factor Villa and Factor IXa. The cloning of the cDNA for Factor VII (U.S. Pat. No. 4,784,950) made it possible to develop activated Factor VII as a pharmaceutical. Factor VIIa was successfully administered for the first time in 1988.

FVII is a single-chain glycoprotein with a molecular weight of about 50 kDa, which is secreted by liver cells into the blood stream as an inactive zymogen of 406 amino acids. It contains 10 γ-carboxy-glutamic acid residues (positions 6, 7, 14, 16, 19, 20, 25, 26, 29, and 35) localized in the N-terminal Gla-domain of the protein. The Gla residues require vitamin K for their biosynthesis. Located C-terminal to the Gla domain are two epidermal growth factor domains followed by a trypsin-type serine protease domain. Further posttranslational modifications of FVII encompass hydroxylation (Asp 63), N-(Asn145 and Asn322) as well as O-type glycosylation (Ser52 and Ser60).

FVII is converted to its active form Factor VIIa by proteolysis of the single peptide bond at Arg152-Ile153 leading to the formation of two polypeptide chains, a N-terminal light chain (24 kDa) and a C-terminal heavy chain (28 kDa), which are held together by one disulfide bridge. In contrast to other vitamin K-dependent coagulation factors no activation peptide, which is cleaved off during activation of these other vitamin-K dependent coagulation factors has been described for FVII. The Arg152-Ile153 cleavage site and some amino acids downstream show homology to the activation cleavage site of other vitamin K-dependent polypeptides.

Essential for attaining the active conformation of Factor VIIa is the formation of a salt bridge after activation cleavage between Ile153 and Asp343. Activation cleavage of Factor VII can be achieved in vitro by Factor Xa, Factor XIIa, Factor IXa, Factor VIIa, Factor Seven Activating Protease (FSAP) and thrombin. Mollerup et al., 1995 (Biotechnol. Bioeng. 48:501-505) reported that some cleavage also occurs in the heavy chain at Arg290 and or Arg315.

NovoSeven® (Novo Nordisk) is a recombinant Factor VIIa product that is approved in both the United States and Europe to treat bleeding in CHwI (Auerswald G and Morfini M, 2010, JCD; 2:(1): 1-8). However, NovoSeven® has an extremely short half-life (2.89 hours in the non-bleeding state and 2.30 hours in the bleeding episodes) so it is recommended that rFVIIa is injected at 2-hourly intervals during the initial treatment of an acute bleeding episode (Brackmann H-H, et al., 2000 (Blood Coagulation and Fibrinolysis. 11(suppl 1):S39-S44).

The clinical use of rFVIIa has been hampered by its extremely short half-life. The need for frequent intravenous (IV) injections carries a significant burden for patients and the physicians treating their disorder. Especially in younger children such a regimen often (but not always), requires the insertion of a venous access device that must be kept extremely clean to avoid infectious complications and prevent the development of clots in the line. The risk and morbidity associated with such devices may prevent some very young children with CHwI from receiving adequate care.

There have been several attempts to use rFVIIa as a prophylactic agent for CHwI (Bianco R P, et al., 2010. Medicina (Buenos Aires). 70: 209-214; Auerswald G and Morfini M, 2010. JCD. 2(1):1-8). A FVIIa product with a prolonged half-life and better recovery rate would allow patients to achieve adequate hemostasis with fewer injections. One of the many benefits of prophylaxis is a decreased annual number of joint bleeds, and, in consequence, a reduced incidence of crippling joint disease. Various studies have been undertaken to provide half-life extended version of FVIIa (see WO2004/101740, WO2007/090584, WO2007/022512, WO2010/091122 and WO2011/092242).

WO2007/090584 relates to Factor VII and Factor VIIa albumin linked polypeptides. These albumin fusions were shown to retain Factor VII/FVIIa biological activity and displayed a significant extension of the functional plasma half-life of Factor VII/VIIa in vivo. U52014/0004095 also relates to FVIIa-albumin fusion proteins.

Ljung R, et al., 2013(Journal of Thrombosis and Haemostasis. 11:1260-1268) showed that multiple doses of a 40K glycoPEGylated recombinant Factor VIIa (rFVIIa) bypassing agent (N7-FP), with a prolonged half-life compared with rFVIIa, was well tolerated in patients with CHwI, and no serious adverse effects were observed. However, although an overall reduction in the number of bleeding events was seen, no dose-response relationship could be determined, i.e., the efficacy of the high dose could tested doses. Consequently, the clinical development of N7-GP in prophylaxis of inhibitor patients was discontinued.

Mahlangu J N, et al., 2012 (Journal of Thrombosis and Haemostasis. 10:773-780) published that a single dose of a variant human recombinant Factor VIIa variant developed for high procoagulant activity and longer action (BAY 86-6150) showed no safety concerns in non-bleeding men with moderate or severe hemophilia A or B with or without inhibitors. The half-life of BAY 86-6150 was 5-7 hours, longer than for rFVIIa. However, the Phase II/III trial was discontinued since neutralizing antibodies against the variant rFVIIa were detected in the trial.

FVIIa polypeptides can also be used as therapy to treat bleeding associated with perioperative and traumatic blood loss in subjects with normal coagulation systems. For example, FVIIa polypeptides can be administered to a patient to promote coagulation and reduce blood loss associated with trauma and surgery and, further, reduce the requirement for blood transfusion.

FVIIa polypeptides can also be used to promote coagulation and prevent blood loss in subjects who have bleeding as a result of traumatic injury. These patients may or may not have hereditary or acquired hemophilia. However, a phase 3 clinical trial evaluating efficacy and safety of rFVIIa as an adjunct to direct hemostasis in major trauma was terminated early (Hauser C J, et al., 2010. The Journal of TRAUMA® Injury, Infection, and Critical Care. 69(3):489-500).

A need persists for an effective dosing regimen with half-life prolonged FVIIa variants and in particular, a prophylactic dosing regimen employing such variants would be highly advantageous.

The present invention relates to dosing regimens with half-life extended Factor VIIa (FVIIa) comprising a FVIIa portion and a half-life enhancing moiety (HLEM) (e.g., rFVIIa-albumin) for use in preventing bleeding in a subject (“prophylactic treatment”), treating a bleeding episode in a subject (“on-demand treatment”), including after trauma, as well as preventing a bleeding episode during or after surgery in patients with or without congenital or acquired bleeding disorders.

In particular, the present invention relates to prophylactic treatment using half-life extended FVIIa. Such prophylactic treatment alleviates discomfort for patients and reduces the number of required visits to a medical professional. These advantages will positively affect patient compliance and thus the effectiveness of prophylactic therapy for hemophilia. Effective prophylaxis may result in a decreased annual number of joint bleeds, and, in consequence, a reduce incidence and/or delay the onset of crippling joint disease.

### SUMMARY OF THE INVENTION

An important aspect of the invention is to provide a regimen for prophylactic treatment of bleeding with a half-life extended FVIIa protein.

The invention relates to a half-life extended FVIIa protein comprising
a) a Factor VIIa (FVIIa) portion, and
b) a half-life enhancing moiety (HLEM)
   for use in a method of preventing bleeding in a subject (“prophylactic treatment”), wherein the half-life extended FVIIa protein is to be administered to the subject at a dose leading to a Cmax of at least about 30 IU per ml of blood, at a dosing interval of at least once every day, wherein the activity (IU) is determined with the Staclot® assay. In a preferred embodiment, the dose of the half-life extended FVIIa protein leads to a Cmax of about 30-160 IU per ml of blood. In another preferred embodiment, the dose of the half-life extended FVIIa protein leads to a Cmax of about 35-130 IU per ml of blood. In still another preferred embodiment, the dose of the half-life extended FVIIa protein leads to a Cmax of about 40-105 IU per ml of blood. In still another preferred embodiment, the dose of the half-life extended FVIIa protein leads to a Cmax of about 46-92 IU per ml of blood. In another preferred embodiment, the dose of the half-life extended FVIIa protein leads to a Cmax of about 30-70 IU per ml of blood. In a more preferred embodiment, the dose of the half-life extended FVIIa protein leads to a Cmax of about 36-56 IU per ml of blood. In another more preferred embodiment, the dose of the half-life extended FVIIa protein leads to a Cmax of about 41-51 IU per ml of blood. In another preferred embodiment, the dose of the half-life extended FVIIa protein leads to a Cmax of about 70-110 IU per ml of blood. In another preferred embodiment, the dose of the half-life extended FVIIa protein leads to a Cmax of about 80-105 IU per ml of blood. In a more preferred embodiment, the dose of the half-life extended FVIIa protein leads to a Cmax of about 82-102 IU per ml of blood. In still another more preferred embodiment, the dose of the half-life extended FVIIa protein leads to a Cmax of about 87-97 IU per ml of blood.

The invention also relates to a half-life extended FVIIa protein comprising
a) a Factor VIIa (FVIIa) portion, and
b) a half-life enhancing moiety (HLEM)
   for use in a method of preventing bleeding in a subject (“prophylactic treatment”), wherein the dose of the FVIIa portion of said half-life extended FVIIa protein to be administered to the subject is about 200-800 μg/kg, at a dosing interval of at least once every day. In a preferred embodiment, the dose of the FVIIa portion of the half-life extended FVIIa protein is about 300-700 μg/kg. In another preferred embodiment, the dose of the FVIIa portion of the half-life extended FVIIa protein is about 320-650 μg/kg. In a more preferred embodiment, the dose of the FVIIa portion of the half-life extended FVIIa protein is about 310-330 μg/kg. In another more preferred embodiment, the dose of the FVIIa portion of the half-life extended FVIIa protein is about 630-650 μg/kg.

The invention further relates to a half-life extended FVIIa protein comprising
a) a Factor VIIa (FVIIa) portion, and
b) a half-life enhancing moiety (HLEM)
   for use in a method of preventing bleeding in a subject (“prophylactic treatment”), wherein the dose of the FVIIa portion of said half-life extended FVIIa protein to be administered to the subject is about 1750-8000 IU/kg, at a dosing interval of at least once every day, wherein the activity (IU) is determined with the Staclot® assay. In a preferred embodiment, the dose of the FVIIa portion of the half-life extended FVIIa protein is about 1800-7000 IU/kg. In another preferred embodiment, the dose of the FVIIa portion of the half-life extended FVIIa protein is about 2500-6500 IU/kg. In a further preferred embodiment, the dose of the FVIIa portion is about 3200-5800 IU/kg. In a more preferred embodiment, the dose of the FVIIa portion of the half-life extended FVIIa protein is about 2800-3200 IU/kg. In another more preferred embodiment, the dose of the FVIIa portion of the half-life extended FVIIa protein is about 5800-6200 IU/kg.

In any one of the above embodiments, the dosing interval may be about once every 1 to 5 days, including once every 1 to 4 days, once every 1 to 3 days, preferably about once every 2 to 4 days or about once every 2 to 3 days. The dosing interval is most preferably about once every other day (i.e., once every 2 days).

In the above embodiments, the method preferably involves a prophylactic dosing regimen.

The invention relates to a half-life extended FVIIa protein comprising
a) a Factor VIIa (FVIIa) portion, and
b) a half-life enhancing moiety (HLEM)
   for use in a method of treating a bleeding episode in a subject (“on-demand treatment”), wherein the half-life extended FVIIa protein is to be administered to the subject at a dose leading to a Cmax of at least about 30 IU per ml of blood, wherein the activity (IU) is determined with the Staclot® assay. In a preferred embodiment, the dose of the half-life extended FVIIa protein leads to a Cmax of about 30-160 IU per ml of blood. In another preferred embodiment, the dose of the half-life extended FVIIa protein leads to a Cmax of about 35-130 IU per ml of blood. In still another preferred embodiment, the dose of the half-life extended FVIIa protein leads to a Cmax of about 40-105 IU per ml of blood. In still another preferred embodiment, the dose of the half-life extended FVIIa protein leads to a Cmax of about 46-92 IU per ml of blood. In another preferred embodiment, the dose of the half-life extended FVIIa protein leads to a Cmax of about 30-70 IU per ml of blood. In a more preferred embodiment, the dose of the half-life extended FVIIa protein leads to a Cmax of about 36-56 IU per ml of blood. In another more preferred embodiment, the dose of the half-life extended FVIIa protein leads to a Cmax of about 41-51 IU per ml of blood. In another preferred embodiment, the dose of the half-life extended FVIIa protein leads to a Cmax of about 70-110 IU per ml of blood. In another preferred embodiment, the dose of the half-life extended FVIIa protein leads to a Cmax of about 80-105 IU per ml of blood. In a more preferred embodiment, the dose of the half-life extended FVIIa protein leads to a Cmax of about 82-102 IU per ml of blood. In still another more preferred embodiment, the dose of the half-life extended FVIIa protein leads to a Cmax of about 87-97 IU per ml of blood. In a preferred embodiment, the first administration leads to termination of bleeding in at least 30% of patients. Optionally, a second administration of the half-life extended FVIIa protein is administered to the subject at a dose equal to the first dose, at a dosing interval of about 5-10 hours. The dosing interval is preferably about 6-8 hours. In a preferred embodiment, the second administration leads to termination of bleeding in at least 60% of patients.

The invention also relates a half-life extended FVIIa protein comprising
a) a Factor VIIa (FVIIa) portion, and
b) a half-life enhancing moiety (HLEM)
   for use in a method of treating a bleeding episode in a subject (“on-demand treatment”), wherein the dose of the FVIIa portion of said half-life extended FVIIa protein to be administered to the subject is about 200-800 μg/kg. In a preferred embodiment, the dose of the FVIIa portion of the half-life extended FVIIa protein is about 300-700 μg/kg. In another preferred embodiment, the dose of the FVIIa portion of the half-life extended FVIIa protein is about 320-650 μg/kg. In a more preferred embodiment, the dose of the FVIIa portion of the half-life extended FVIIa protein is about 310-330 μg/kg. In another more preferred embodiment, the dose of the FVIIa portion of the half-life extended FVIIa protein is about 630-650 μg/kg. In a preferred embodiment, the first administration leads to termination of bleeding in at least 30% of patients. Optionally, a second administration of the half-life extended FVIIa protein is administered to the subject at a dose equal to the first dose, at a dosing interval of about 5-10 hours. The dosing interval is preferably about 6-8 hours. In a preferred embodiment, the second administration leads to termination of bleeding in at least 60% of patients.

The invention further relates to a half-life extended FVIIa protein comprising
a) a Factor VIIa (FVIIa) portion, and
b) a half-life enhancing moiety (HLEM)
   for use in a method of treating a bleeding episode in a subject (“on-demand treatment”), wherein the dose of the FVIIa portion of said half-life extended FVIIa protein to be administered to the subject is about 1750-8000 IU/kg, wherein the activity (IU) is determined with the Staclot® assay. In a preferred embodiment, the dose of the FVIIa portion of the half-life extended FVIIa protein is about 1800-7000 IU/kg. In another preferred embodiment, the dose of the FVIIa portion of the half-life extended FVIIa protein is about 2500-6500 IU/kg. In a further preferred embodiment, the dose of the FVIIa portion is about 3200-5800 IU/kg. In a more preferred embodiment, the dose of the FVIIa portion of the half-life extended FVIIa protein is about 2800-3200 IU/kg. In another more preferred embodiment, the dose of the FVIIa portion of the half-life extended FVIIa protein is about 5800-6200 IU/kg. In a preferred embodiment, the first administration leads to termination of bleeding in at least 30% of patients. Optionally, a second administration of the half-life extended FVIIa protein is administered to the subject at a dose equal to the first dose, at a dosing interval of about 5-10 hours. The dosing interval is preferably about 6-8 hours. In a preferred embodiment, the second administration leads to termination of bleeding in at least 60% of patients.

The invention relates to a half-life extended FVIIa protein comprising
a) a Factor VIIa (FVIIa) portion, and
b) a half-life enhancing moiety (HLEM)
   for use in a method of treating a bleeding episode in a subject which is a result of trauma, wherein the half-life extended FVIIa protein is to be administered to the subject at a dose leading to a Cmax of at least about 30 IU per ml of blood, wherein the activity (IU) is determined with the Staclot® assay. In a preferred embodiment, the dose of the half-life extended FVIIa protein leads to a Cmax of about 30-160 IU per ml of blood. In another preferred embodiment, the dose of the half-life extended FVIIa protein leads to a Cmax of about 35-130 IU per ml of blood. In still another preferred embodiment, the dose of the half-life extended FVIIa protein leads to a Cmax of about 40-105 IU per ml of blood. In still another preferred embodiment, the dose of the half-life extended FVIIa protein leads to a Cmax of about 46-92 IU per ml of blood. In another preferred embodiment, the dose of the half-life extended FVIIa protein leads to a Cmax of about 30-70 IU per ml of blood. In a more preferred embodiment, the dose of the half-life extended FVIIa protein leads to a Cmax of about 36-56 IU per ml of blood. In another more preferred embodiment, the dose of the half-life extended FVIIa protein leads to a Cmax of about 41-51 IU per ml of blood. In another preferred embodiment, the dose of the half-life extended FVIIa protein leads to a Cmax of about 70-110 IU per ml of blood. In another preferred embodiment, the dose of the half-life extended FVIIa protein leads to a Cmax of about 80-105 IU per ml of blood. In a more preferred embodiment, the dose of the half-life extended FVIIa protein leads to a Cmax of about 82-102 IU per ml of blood. In still another more preferred embodiment, the dose of the half-life extended FVIIa protein leads to a Cmax of about 87-97 IU per ml of blood. In a preferred embodiment, the first administration leads to a reduction in transfusion requirements of red blood cells (RBC) and/or fresh frozen plasma (FFP) by at least 20% within 24 hours after administration.

The invention also relates a half-life extended FVIIa protein comprising
a) a Factor VIIa (FVIIa) portion, and
b) a half-life enhancing moiety (HLEM)
   for use in a method of treating a bleeding episode in a subject which is a result of trauma, wherein the dose of the FVIIa portion of said half-life extended FVIIa protein to be administered to the subject is about 200-800 μg/kg. In a preferred embodiment, the dose of the FVIIa portion of the half-life extended FVIIa protein is about 300-700 μg/kg. In another preferred embodiment, the dose of the FVIIa portion of the half-life extended FVIIa protein is about 320-650 μg/kg. In a more preferred embodiment, the dose of the FVIIa portion of the half-life extended FVIIa protein is about 310-330 μg/kg. In another more preferred embodiment, the dose of the FVIIa portion of the half-life extended FVIIa protein is about 630-650 μg/kg. In a preferred embodiment, the first administration leads to a reduction in transfusion requirements (RBC and/or FFP) by 20% within 24 h after administration.

The invention further relates to a half-life extended FVIIa protein comprising
a) a Factor VIIa (FVIIa) portion, and
b) a half-life enhancing moiety (HLEM)
   for use in a method of treating a bleeding episode in a subject which is a result of trauma, wherein the dose of the FVIIa portion of said half-life extended FVIIa protein to be administered to the subject is about 1750-8000 IU/kg, wherein the activity (IU) is determined with the Staclot® assay. In a preferred embodiment, the dose of the FVIIa portion of the half-life extended FVIIa protein is about 1800-7000 IU/kg. In another preferred embodiment, the dose of the FVIIa portion of the half-life extended FVIIa protein is about 2500-6500 IU/kg. In a further preferred embodiment, the dose of the FVIIa portion is about 3200-5800 IU/kg. In a more preferred embodiment, the dose of the FVIIa portion of the half-life extended FVIIa protein is about 2800-3200 IU/kg. In another more preferred embodiment, the dose of the FVIIa portion of the half-life extended FVIIa protein is about 5800-6200 IU/kg. In a preferred embodiment, the first administration leads to a reduction in transfusion requirements (RBC and/or FFP) by 20% within 24 h after administration.

The invention relates to a half-life extended FVIIa protein comprising
a) a Factor VIIa (FVIIa) portion, and
b) a half-life enhancing moiety (HLEM)
   for use in a method of preventing a bleeding episode in a subject during or after surgery, wherein the half-life extended FVIIa protein is to be administered to the subject before, during and/or after the surgery at a dose leading to at least a Cmax of about 30 IU per ml of blood, wherein the activity (IU) is determined with the Staclot® assay. In a preferred embodiment, the dose of the half-life extended FVIIa protein leads to a Cmax of about 30-160 IU per ml of blood. In another preferred embodiment, the dose of the half-life extended FVIIa protein leads to a Cmax of about 35-130 IU per ml of blood. In still another preferred embodiment, the dose of the half-life extended FVIIa protein leads to a Cmax of about 40-105 IU per ml of blood. In still another preferred embodiment, the dose of the half-life extended FVIIa protein leads to a Cmax of about 46-92 IU per ml of blood. In another preferred embodiment, the dose of the half-life extended FVIIa protein leads to a Cmax of about 30-70 IU per ml of blood. n a more preferred embodiment, the dose of the half-life extended FVIIa protein leads to a Cmax of about 36-56 IU per ml of blood. In another more preferred embodiment, the dose of the half-life extended FVIIa protein leads to a Cmax of about 41-51 IU per ml of blood. In another preferred embodiment, the dose of the half-life extended FVIIa protein leads to a Cmax of about 70-110 IU per ml of blood. In another preferred embodiment, the dose of the half-life extended FVIIa protein leads to a Cmax of about 80-105 IU per ml of blood. In a more preferred embodiment, the dose of the half-life extended FVIIa protein leads to a Cmax of about 82-102 IU per ml of blood. In still another more preferred embodiment, the dose of the half-life extended FVIIa protein leads to a Cmax of about 87-97 IU per ml of blood. The half-life extended FVIIa protein maybe re-administered to the subject at a dose equal to the first dose, at a dosing interval of about 5-10 hours, preferably about 6-8 hours. Alternatively, the half-life extended FVIIa protein may be used in a continuous infusion during the surgery.

The invention also relates a half-life extended FVIIa protein comprising
a) a Factor VIIa (FVIIa) portion, and
b) a half-life enhancing moiety (HLEM)
   for use in a method of preventing a bleeding episode in a subject during or after surgery, wherein the half-life extended FVIIa protein is to be administered to the subject before, during and/or after the surgery, wherein the dose of of the FVIIa portion of said half-life extended FVIIa protein to be administered to the subject is about 200-800 μg/kg. In a preferred embodiment, the dose of the FVIIa portion of the half-life extended FVIIa protein is about 300-700 μg/kg. In another preferred embodiment, the dose of the FVIIa portion of the half-life extended FVIIa protein is about 320-650 μg/kg. In a more preferred embodiment, the dose of the FVIIa portion of the half-life extended FVIIa protein is about 310-330 μg/kg. In another more preferred embodiment, the dose of the FVIIa portion of the half-life extended FVIIa protein is about 630-650 μg/kg. Preferably, the half-life extended FVIIa protein is to be readministered to the subject at a dose equal to the first dose, at a dosing interval of about 5-10 hours, preferably about 6-8 hours. Alternatively, the half-life extended FVIIa protein may be used in a continuous infusion during the surgery.

The invention further relates to a half-life extended FVIIa protein comprising
a) a Factor VIIa (FVIIa) portion, and
b) a half-life enhancing moiety (HLEM)
   for use in a method of preventing a bleeding episode in a subject during or after surgery, wherein the half-life extended FVIIa protein is to be administered to the subject before, during and/or after the surgery, wherein the dose of the FVIIa portion of said half-life extended FVIIa protein to be administered to the subject is about 1750-8000 IU/kg, wherein the activity (IU) is determined with the Staclot® assay. In a preferred embodiment, the dose of the FVIIa portion of the half-life extended FVIIa protein is about 1800-7000 IU/kg. In another preferred embodiment, the dose of the FVIIa portion of the half-life extended FVIIa protein is about 2500-6500 IU/kg. In a further preferred embodiment, the dose of the FVIIa portion is about 3200-5800 IU/kg. In a more preferred embodiment, the dose of the FVIIa portion of the half-life extended FVIIa protein is about 2800-3200 IU/kg. In another more preferred embodiment, the dose of the FVIIa portion of the half-life extended FVIIa protein is about 5800-6200 IU/kg. Preferably, the half-life extended FVIIa protein is to be readministered to the subject at a dose equal to the first dose, at a dosing interval of about 5-10 hours, preferably about 6-8 hours. Alternatively, the half-life extended FVIIa protein may be used in a continuous infusion during the surgery.

In preferred embodiments, the hemostatic potential is maintained at a trough of at least about 1%, preferably at least about 2% above baseline for the entire dosing interval, and more preferably between 5 and 15% above baseline for the entire dosing interval.

The half-life extended FVIIa protein of the present invention preferably has a half-life of greater than about 5 hours.

In any one of the above embodiments, the half-life enhancing moiety (HLEM) may be a polyalkylene glycol moiety, preferably a PEG. Alternatively, the half-life enhancing moiety (HLEM) is a half-life enhancing polypeptide (HLEP). In one embodiment, the half-life enhancing polypeptide (HLEP) is a carboxy-terminal peptide (CTP). In another embodiment, the half-life enhancing polypeptide (HLEP) is an FcRn binding partner. Preferably, the half-life enhancing polypeptide (HLEP) FcRn binding partner is albumin or an immunoglobulin without an antigen binding domain (e.g., Fc).

Most preferably, the half-life enhancing polypeptide (HLEP) is albumin. In this embodiment, the dose of the half-life extended FVIIa protein is at least about 500 μg/kg. In a preferred embodiment, the dose of the half-life extended FVIIa protein is about 500-2500 μg/kg. In another preferred embodiment, the dose of the half-life extended FVIIa protein is about 750-2000 μg/kg. In still another preferred embodiment, the dose of the half-life extended FVIIa protein is about 1000-1500 μg/kg. In another preferred embodiment, the dose of the half-life extended FVIIa protein is about 1100-1600 μg/kg. In still another preferred embodiment, the dose of the half-life extended FVIIa protein is about 1200-1500 μg/kg. In still another preferred embodiment, the dose of the half-life extended FVIIa protein is about 1300-1500 μg/kg. In still another preferred embodiment, the dose of the half-life extended FVIIa protein is about 1400-1500 μg/kg. In a more preferred embodiment, the dose of the half-life extended FVIIa protein is about 740-760 μg/kg. In another more preferred embodiment, the dose of the half-life extended FVIIa protein is about 1490-1510 μg/kg. For use in a method of preventing bleeding in a subject (“prophylactic treatment”), the dosing interval may be about once every 1 to 5 days, including once every 1 to 4 days, once every 1 to 3 days, preferably about once every 2 to 4 days or about once every 2 to 3 days, most preferably about once every other day (i.e., once every 2 days). For use in a method of treating a bleeding episode in a subject (“on-demand treatment”), a second administration of the half-life extended FVIIa protein may be administered to the subject at a dose equal to the first dose, at a dosing interval of about 5-10 hours, preferably about 6-8 hours. For use in a method of preventing a bleeding episode in a subject during or after surgery, the half-life extended FVIIa protein may be readministered to the subject at a dose equal to the first dose, at a dosing interval of about 5-10 hours, preferably about 6-8 hours.

As discussed above, it would be of a particular advantage to extend the treatment interval with respect to the administration of FVIIa variants. Thus, in another aspect, the invention relates to a half-life extended Factor VIIa (FVIIa) protein comprising
a) a FVIIa portion, and
b) a half-life enhancing polypeptide (HLEP)
   for use in a method of preventing bleeding in a subject at a dosing interval of about once every 2 to 4 days. In one embodiment, the half-life enhancing polypeptide (HLEP) is a carboxy-terminal peptide (CTP). In another embodiment, the half-life enhancing polypeptide (HLEP) is an FcRn binding partner. Preferably, the half-life enhancing polypeptide (HLEP) FcRn binding partner is albumin or an immunoglobulin without an antigen binding domain (e.g., Fc). In a more preferred embodiment the half-life enhancing polypeptide (HLEP) is albumin. The dosing interval may be about once every 1 to 5 days, including once every 1 to 4 days, once every 1 to 3 days. Preferably the extended dosing interval is about once every 2 to 4 days or about once every 2 to 3 days. The dosing interval is most preferably about once every other day (i.e., once every 2 days). In this embodiment, the method preferably involves a prophylactic dosing regimen.

For the purposes of the invention, the preferred subject to be administered the half-life extended FVIIa protein is human. Particularly preferred is a human that suffers from hemophilia A or hemophilia B and especially a hemophilia A or hemophilia B patient that has developed inhibitors (antibodies) against FVIII and/or FIX (i.e., Congenital Hemophilia with Inhibitors, CHwI). In another preferred embodiment, the human suffers from acquired hemophilia. The acquired hemophilia can be acquired hemophilia A or B. In still another embodiment, the human suffers from inherited Factor VII deficiency. In a particularly preferred embodiment, the dose is to be administered intravenously.

In any of the treatment methods of the invention, the subject preferably has inhibitory antibodies against FVIII and/or FIX. The subject preferably has Congenital Hemophilia with Inhibitors (CHwI). The use of half-life extended FVIIa according to the invention is particularly advantageous in such patients.

Notably, in the treatment of trauma, it is not required that the subject has a congenital or acquired bleeding disorder. With respect to treating trauma, the invention contemplates administering the half-life extended FVIIa protein to patients without a preexisting bleeding disorder, such as car accident victims or soldiers injured in combat.

The half-life extended FVIIa protein of the invention preferably has the sequence set forth in SEQ ID NO: 1. The underlined amino acid sequence RI in the bold sequence is cleaved behind the amino acid R when FVII is activated to FVIIa (the molecule still being held together by disulfide-bridges). The FVIIa part (in bold) is followed by SSGGSGGSGGSGGSGGSGGSGGSGGSGGSGS (which is a 31 aa flexible linker) followed then from DAHHK to the C-terminal end by human albumin (full length).

| | | |
|---|---|---|
| 1 | ANAFLEELFPGSLERECKEECCSFEEAREI FKDAERTKLFW SYSDGDCCASSPCCNGGS | 60 |
| 61 | CKDQLQSYI CFCLPAFEGRNCETHKDDQLI CVNENGGCEQYCSDHTGTKRSCRCHEGYSL | 120 |
| 121 | LADGVSCTPTVEYPCGKI PI LEKRNASKPQGRI VGGKVCPKGECPWQVLLLVNGAQLCGG | 180 |
| 181 | TLI NTI WVVSAAHCFDKI KNWRNLI AVLGEHDLSEHDGDEQSRRVAQVI I PSTYVPGTTN | 240 |
| 241 | HDI ALLFLHCPVVLTDHVVPLCLPERTFSERTLAFVFFSLVSGWGQLLDRGATALELMVL | 300 |
| 301 | NVPRLMTQDCLQQSRKVGDSPN TEYMFCAGYSDGSKDSCKGDSGGPHATHYRGTWLTG | 360 |
| 361 | I VSWGQGCATVGHFGVYTRVSQYI EWLQKLMRSEPRPGVLLRAPFPSSGGSGGSGGSGGS | 420 |
| 421 | GGSGGSGGSGGSGGSGSDAHKSEVAVHRFKDLGEENFKALVLIAFAQYLQQCPFEDHVKLV | 480 |
| 481 | NEVTEFAKTCVADESAENCDKSLHTLFGDKLCTVATLRETYGEMADCCAKQEPERNECFL | 540 |
| 541 | QHKDDNPNLPRLVRPEVDVMCTAFHDNEETFLKKYLYEIARRHPYFYAPELLFFAKRYKA | 600 |
| 601 | AFTECCQAADKAACLLPKLDELRDEGKASSAKQRLKCASLQKFGERAFKAWAVARLSQRF | 660 |
| 661 | PKAEFAEVSKLVTDLTKVHTECCHGDLLECADDRADLAKYICENQDSISSKLKECCEKPL | 720 |
| 721 | LEKSHCIAEVENDEMPADLPSLAADFVESKDVCKNYAEAKDVFLGMFLYEYARRHPDYSV | 780 |
| 781 | VLLLRLAKTYETTLEKCCAAADPHECYAKVFDEFKPLVEEPQNLIKQNCELFEQLGEYKF | 840 |
| 841 | QNALLVRYTKKVPQVSTPTLVEVSRNLGKVGSKCCKHPEAKRMPCAEDYLSVVLNQLCVL | 900 |
| 901 | HEKTPVSDRVTKCCTESLVNRRPCFSALEVDETYVPKEFNAETFTFHADICTLSEKERQI | 960 |
| 961 | KKQTALVELVKHKPKATKEQLKAVMDDFAAFVEKCCKADDKETCFAEEGKKLVAASQAAL | 1020 |
| 1021 | GL | 1022 |

Alternatively, the sequence of the fusion protein has at least 70% identity to the sequence set forth in SEQ ID NO: 1. The sequence of the fusion protein may have at least 75% identity to the sequence set forth in SEQ ID NO: 1. The sequence of the fusion protein may have at least 80% percent identity to the sequence set forth in SEQ ID NO: 1. The sequence of the fusion protein may have at least 85% percent identity to the sequence set forth in SEQ ID NO: 1. The sequence of the fusion protein may have at least 90% percent identity to the sequence set forth in SEQ ID NO: 1. The sequence of the fusion protein may have at least 95% percent identity to the sequence set forth in SEQ ID NO: 1. The sequence of the fusion protein may have at least 98% percent identity to the sequence set forth in SEQ ID NO: 1. The sequence of the fusion protein may have at least 98% percent identity to the sequence set forth in SEQ ID NO: 1.

### DESCRIPTION OF FIGURES

FIG. 1: CONSORT diagram showing flow and allocation of study participants through the study.

FIG. 2: Figure showing baseline corrected FVIIa plasma levels in subjects receiving rVIIa-FP over time per dose group.

FIG. 3: Study procedures for hemodilution, treatment, experimental kidney trauma and assessment of hemostatic effect. Abbreviations: HES, hydroxyethyl starch; rVIIa-FP (CSL689 (SEQ ID NO: 1)), fusion protein linking activated Factor VIIa with human albumin; rFVIIa (NovoSeven®) activated recombinant Factor VII.

FIG. 4: Total blood loss following standardized kidney injury in hemodiluted rabbits following treatment with saline (positive control), CSL689 (SEQ ID NO: 1) (rVIIa-FP) or NovoSeven® (rFVIIa).

FIG. 5: Time to hemostasis following standardized kidney injury in hemodiluted rabbits following treatment with saline (positive control), CSL689 (SEQ ID NO: 1) (rVIIa-FP) or NovoSeven® (rFVIIa).

### DETAILED DESCRIPTION OF THE INVENTION

The present invention provides prophylactic, “on-demand” and surgical dosing regimens for a half-life extended FVIIa protein, comprising preferably the HLEP albumin wherein the Factor VIIa (FVIIa) portion is connected to the albumin via a 31 amino acid poly glycine-serine (GS) non-cleavable peptide linker. The dosing interval can be at least once every day, such as every other day, but even longer periods of prophylactic dosing can be achieved than previously envisioned, such as once every 3, 4 or 5 days.

The longer half-life and prolonged biological activity of half-life extended FVIIa protein (e.g., rVIIa-albumin) compared to other known FVIIa products surprisingly allows for prophylactic treatment of hemophilia with dosing intervals that are significantly longer than suggested by the prior art for rFVIIa (e.g., NovoSeven®).

CSL689 (SEQ ID NO: 1) (also referred to herein as “rVIIa-albumin” or “rVIIa-FP”) is a purified recombinant protein comprising of human coagulation factor VII (FVII) in its activated form (FVIIa) linked to human albumin by a 31 amino acid poly glycine-serine (GS) linker peptide. Unlike some other molecules that have been evaluated, no amino acids were exchanged to increase the potency or prolong the half-life of FVIIa so that it is essentially wild-type FVIIa fused via a non-cleavable linker to human albumin. A phase I study has been completed which examined a single dose of CSL689 in healthy adult male subjects anticoagulated with warfarin (Golor G, et al., 2013. Journal of Thrombosis and Haemostasis. 11:1977-1985). The results demonstrate that both the half-life and the biological activity of FVIIa is prolonged. It has an extended half-life of 6.1 to 9.7 h (3-4 times increased).

The technical advantage of the present invention is that the half-life extended FVIIa protein (e.g., rVIIa-albumin/ rVIIa-FP) has both a longer half-life and a prolonged biological activity than other known FVIIa products. This surprisingly allows for prevention and treatment of hemophilia with dosing intervals that are significantly longer than suggested by the prior art for rFVIIa. Less frequent administrations are required to achieve the same FVIIa peak and trough activity levels in a patient.

In preferred embodiments, the hemostatic potential is maintained at a trough of at least about 0.5%, or at least about 1%, or at least about 2%, or at least about 3%, or at least about 4% above baseline for the entire dosing interval, preferably between 5 and 15% above baseline for the entire dosing interval.

The improved properties of the half-life extended FVIIa also improves the efficacy of “on demand treatment” compared to rFVIIa (NovoSeven®) after trauma. These patients may or may not have a congenital or acquired bleeding disorder. This advantage was demonstrated by experiments in a rabbit model of trauma (see Example 3).

The improved properties of the half-life extended FVIIa also improves the efficacy for the treatment of acquired hemophilia, compared to rFVIIa (NovoSeven®). This is supported by experiments in a monkey model (see Example 4). The half-life extended FVIIa proteins provided herein can be administered after trauma or for treating acquired hemophilia with greater efficacy, at lower doses, less frequently, and/or with fewer potential adverse reactions.

### Definitions

“Prophylactic treatment”, as used herein, means administering a half-life extended Factor VIIa protein in multiple doses to a subject over a course of time to increase the level of Factor VIIa activity in a subject's plasma. Preferably, the increased level is sufficient to decrease the incidence of spontaneous bleeding or to prevent bleeding in the event of an unforeseen injury. Prophylactic treatment decreases or prevents bleeding episodes, for example, those described under on-demand treatment. Prophylactic treatment may be fixed or may be individualized, as discussed under “dosing interval”, e.g., to compensate for inter-patient variability.

Prophylaxis was conceived from the observation that moderate hemophilia patients with clotting factor level >1 IU/dl (>1%) seldom experience spontaneous bleeding and have much better preservation of joint function. Therefore, to prevent bleeding and joint destruction, the goal of therapy is to preserve normal musculoskeletal function (GUIDELINES FOR THE MANAGEMENT OF HEMOPHILIA, 2^{nd} edition, Prepared by the Treatment Guidelines Working Group, on behalf of the World Federation of Hemophilia (WFH)). Prophylaxis with FVIIa activity in a patient should be maintained corresponding to the hemostatic potential of above 1% during the entire treatment period. A hemostatic potential of above 1% is in reference to the normal level in a healthy person of the missing or the functionally defect coagulation factor or of the coagulation factor against which inhibitory antibodies are present. As used in the present invention a residual level of 1% of functional FVIII or functional FIX or an inhibition of 99% of the normal FVIII or the normal FIX activity in a patient by inhibitory antibodies would correspond to 1% hemostatic potential.

“On-demand treatment”, as used herein, means administering a half-life extended Factor VIIa protein to control a bleeding episode in a subject. The bleeding episode may occur spontaneously or may be a result of a trauma. The subject may or may not have a congenital or acquired bleeding disorder. Preferably, the administered half-life extended Factor VIIa protein is sufficient to decrease the bleeding. A second administration or multiple administrations of a dose equal to the first dose may be required to control bleeding. On-demand treatment may be fixed or may be individualized, as discussed under “dosing interval”, e.g., to compensate for inter-patient variability.

“Dosing interval”, as used herein, means the amount of time that elapses between multiple doses being administered to a subject. The dosing interval in the methods of the invention using a chimeric FVIIa-HLEM, e.g., FVIIa-HLEP, may be at least about one and one-half to eight times longer than the dosing interval required for an equivalent amount (in IU/kg) of said Factor VIIa without the HLEM, e.g., albumin (i.e., a polypeptide consisting of said FVIIa). The dosing interval when administering, e.g., a half-life extended Factor VIIa protein (e.g., rVIIa-albumin/rVIIa-FP) of the invention may be at least about one and one-half times to eight times longer than the dosing interval required for an equivalent amount of said Factor VIIa without the HLEM, e.g., albumin.

The dosing interval may be at least once every day, about once every 1 to 5 days, about once every 1 to 4 days, about once every 1 to 3 days, preferably about once every 2 to 4 days or about once every 2 to 3 days. In particular, dosing intervals of once every other day (i.e., once every 2 days) is contemplated.

The dosing interval may, alternatively, be an individualized interval that is determined for each subject based on pharmacokinetic data or other information about that subject. The individualized dose/dosing interval combination may be the same as those for fixed interval regimens in the preceding paragraphs, or may differ. The regimen may initially be at a fixed dosing interval, and then it may change to an individualized dosing interval. The regimen may initially be at a fixed dose (e.g., IU/kg or μg/kg) and dosing interval, and then it may change to an individualized dosing interval with the fixed dose. The regimen may also initially be at a fixed dosing interval and dose (e.g., IU/kg or μg/kg), and then it may change to an individualized dose with the same fixed dosing interval.

“Median dose”, as used herein, means half of the study subjects used higher than that dose and half of the study subjects used lower than that dose. “Mean dose” means an average dose (is computed by adding up all the doses and dividing by the total number of the doses). For a given dose, “about” means the dose indicated plus or minus 1, 2, 5, 10, 15 or 20% of that indicated dose. For a dosing interval of about once every 1 to 5 days, “about” means plus or minus 12 hours. For a dosing interval of about once every 1 to 4 days or about once every 2 to 4 days, “about” means plus or minus 10 hours. For a dosing interval of about once every 1 to 3 days or about once every 2 to 3 days, “about” means plus or minus 8 hours. For a dosing interval of about once every other day, or about once every day, “about” means plus or minus 6 hours.

For the purposes of the present invention, “half-life” in the context of administering FVIIa, or a half-life extended FVIIa protein, is defined as the amount of time required for the activity of FVIIa in plasma, as determined with the Staclot® assay, to be reduced by half.

“Cmax” is defined as the maximum plasma FVIIa activity which is measured after administration of FVIIa, as determined with the Staclot® assay.

“Half-life extended” refers to an FVIIa protein which has a longer half-life in comparison with recombinant FVIIa (i.e., NovoSeven®).

The therapeutic dose of the half-life extended FVIIa protein used in the methods of the invention lead to a Cmax of about at least about 30 IU per ml of blood at a dosing interval of at least once every day. Preferably, the dose of the half-life extended FVIIa protein leads to a Cmax of about 30-160 IU per ml of blood. In a preferred embodiment, the dose of the half-life extended FVIIa protein leads to a Cmax of about 35-130 IU per ml of blood. In another preferred embodiment, the dose of the half-life extended FVIIa protein leads to a Cmax of about 40-105 IU per ml of blood. In a highly preferred embodiment, the dose of the half-life extended FVIIa protein leads to a Cmax of about 46-92 IU per ml of blood. In another preferred embodiment, the dose of the half-life extended FVIIa protein leads to a Cmax of about 30-70 IU per ml of blood. In still another preferred embodiment, the dose of the half-life extended FVIIa protein leads to a Cmax of about 36-56 IU per ml of blood. In a more preferred embodiment, the dose of the half-life extended FVIIa protein leads to a Cmax of about 41-51 IU per ml of blood. In another preferred embodiment, the dose of the half-life extended FVIIa protein leads to a Cmax of about 70-110 IU per ml of blood. In still another preferred embodiment, the dose of the half-life extended FVIIa protein a leads to a Cmax of about 80-105 IU per ml of blood. In a more preferred embodiment, the dose of the half-life extended FVIIa protein leads to a Cmax of about 82-102 IU per ml of blood. In still another more preferred embodiment, the dose of the half-life extended FVIIa protein leads to a Cmax of about 87-97 IU per ml of blood. The activity of the half-life extended FVIIa protein is determined with the Staclot® assay.

Alternatively, the therapeutic dose of the FVIIa portion of the half-life extended FVIIa protein used in the methods of the invention is about 200-800 μg/kg at a dosing interval of at least once every day. For the purposes of the invention, a dose in μg/kg refers to μg of the half-life extended FVIIa protein per kg of body weight of the subject. Preferably, the dose of the FVIIa portion of the half-life extended FVIIa protein is about 300-700 μg/kg. In a preferred embodiment, the dose of the FVIIa portion of the half-life extended FVIIa protein is about 320-650 μg/kg. In a more preferred embodiment, the dose of the FVIIa portion of the half-life extended FVIIa protein is about 310-330 μg/kg. In another more preferred embodiment, the dose of the FVIIa portion of the half-life extended FVIIa protein is about 630-650 μg/kg.

As a further alternative, the therapeutic dose of the FVIIa portion of the half-life extended FVIIa protein used in the methods of the invention is about 1750-8000 IU/kg, at a dosing interval of at least once every day. Preferably, the dose of the FVIIa portion of the half-life extended FVIIa protein is about 1800-7000 IU/kg. In a preferred embodiment, the dose of the FVIIa portion of the half-life extended FVIIa protein is about 2500-6500 IU/kg. In a further preferred embodiment, the dose of the FVIIa portion is about 3200-5800 IU/kg. In a more preferred embodiment, the dose of the FVIIa portion of the half-life extended FVIIa protein is about 2800-3200 IU/kg. In another more preferred embodiment, the dose of the FVIIa portion of the half-life extended FVIIa protein is about 5800-6200 IU/kg. The activity of the half-life extended FVIIa protein is determined with the Staclot® assay.

As still a further alternative, the therapeutic dose of a half-life extended FVIIa protein comprising albumin as the half-life enhancing polypeptide (HLEP), used in the methods of the invention, is about 500 μg/kg. In a preferred embodiment, the dose of the half-life extended FVIIa protein is about 500-2500 μg/kg. In a preferred embodiment, the dose of the half-life extended FVIIa protein is about 750-2000 μg/kg. In another preferred embodiment, the dose of the half-life extended FVIIa protein is about 1000-1500 μg/kg. In another preferred embodiment, the dose of the half-life extended FVIIa protein is about 1100-1600 μg/kg. In still another preferred embodiment, the dose of the half-life extended FVIIa protein is about 1200-1500 μg/kg. In still another preferred embodiment, the dose of the half-life extended FVIIa protein is about 1300-1500 μg/kg. In still another preferred embodiment, the dose of the half-life extended FVIIa protein is about 1400-1500 μg/kg. In a more embodiment, the dose of the half-life extended FVIIa protein is about 740-760 μg/kg. In another more preferred embodiment, the dose of the half-life extended FVIIa protein is about 1490-1510 μg/kg.

Preferred doses and dosing intervals are as follows: a dose of the half-life extended FVIIa protein that leads to a Cmax of about 41-51 IU per ml of blood at a dosing interval of about once every other day; a dose of the half-life extended FVIIa protein that leads to a Cmax of about 87-97 IU per ml of blood at a dosing interval of about once every other day; the dose of the FVIIa portion of the half-life extended FVIIa protein is 310-330 μg/kg at a dosing interval of about once every other day; the dose of the FVIIa portion of the half-life extended FVIIa protein is 630-650 μg/kg at a dosing interval of about once every other day; the dose of the FVIIa portion of the half-life extended FVIIa protein is at a dosing interval of 2800-3200 IU/kg about once every other day; and the dose of the FVIIa portion of the half-life extended FVIIa protein is at a dosing interval of 5800-6200 IU/kg about once every other day.

“Factor VII/VIIa” as used in this application means a therapeutic polypeptide consisting of either the non-activated form (Factor VII) or the activated form (Factor VIIa) or mixtures thereof. Factor VII/VIIa within the above definition includes polypeptides that have the amino acid sequence of native human Factor VII/VIIa. It also includes polypeptides with a slightly modified amino acid sequence, for instance, a modified N-terminal or C-terminal end including terminal amino acid deletions or additions as long as those polypeptides substantially retain the biological activity of Factor VIIa. “Factor VII” within the above definition also includes natural allelic variations that may exist and occur from one individual to another. Factor VII within the above definition further includes variants of FVII/FVIIa. Such variants differ in one or more amino acid residues from the wild type sequence. Examples of such differences may include truncation of the N- and/or C-terminus by one or more amino acid residues (e.g. 1 to 10 amino acid residues), or addition of one or more extra residues at the N- and/or C-terminus, as well as conservative amino acid substitutions, i.e. substitutions performed within groups of amino acids with similar characteristics, e.g. (1) small amino acids, (2) acidic amino acids, (3) polar amino acids, (4) basic amino acids, (5) hydrophobic amino acids, and (6) aromatic amino acids. Examples of such conservative substitutions are shown in the following table.

TABLE 1

| | | | | |
|---|---|---|---|---|
| (1) | Alanine | Glycine | | |
| (2) | Aspartic acid | Glutamic acid | | |
| (3a) | Asparagine | Glutamine | | |
| (3b) | Serine | Threonine | | |
| (4) | Arginine | Histidine | Lysine | |
| (5) | Isoleucine | Leucine | Methionine | Valine |
| (6) | Phenylalanine | Tyrosine | Tryptophane | |

The in vivo half-life of the half-life extended FVIIa protein of the invention, in general determined as terminal half-life or β-half-life, is usually at least about 25%, preferably at least about 50%, and more preferably more than 100% higher than the in vivo half-life of the non-fused polypeptide.

The FVIIa activity of a half-life extended FVIIa protein is measured using Staclot®. As used herein, the Staclot® assay is the Staclot®VIIa-rTF (Diagnostica Stago, France, see:
http://www.stago-cn.com/en/products-services/catalogue/reagents/fiche-produit/selection/type-reagents/reference/staclotR-viia-rtf).

Activity is measured in the Staclot® assay after activation of the half-life extended FVIIa protein in International Units (IU) per 100 IU of Factor VII/VIIa antigen as measured by ELISA, based on the method described by Morissey et al., 1993. Blood. 81:734-744.

The term “FVIIa portion” or FVII portion”, as used herein, means the part of a half-life extended FVIIa or FVII molecule which part is derived entirely from the amino acid sequence of native human Factor VII/VIIa or of variants thereof as defined under “FVII/VIIa” and is not derived from the half-life extending moiety. By way of non-limiting example in SEQ ID NO: 1 the “FVIIa portion” is the polypeptidic chain from position 1 to position 406 (including position 1 and 406).

The term “dose of the FVIIa portion”, as used herein, means the weight of the “FVII portion” in a dose of a half-life extended FVIIa molecule per kg bodyweight of the subject (e.g., a human person) receiving the dose. For example in CSL689 (SEQ ID NO:1) the FVIIa portion in CSL 689 has a molecular weight of 51000 Da. The linker and the albumin of CSL689 have a molecular weight of 68.689 Da. Therefore the FVIIa portion of CSL689 is 42.6%. Therefore a dose of 750 μg/kg of CSL689 corresponds to 320 μg/kg for the FVIIa portion and a dose of 1500 μg/kg of CSL689 corresponds to 640 μg/kg for the FVIIa portion.

The functional half-life in vivo of the wild type form of human Factor VIIa is approximately 2 hours in humans. The functional half-life of the Factor VIIa linked albumin polypeptides of the invention is usually at least about 4 hours, preferably at least about 6 hours, more preferably at least about 12 hours.

“Half-life enhancing moiety” (HLEM), as used in this application, means any moiety that extends the half-life of FVIIa. The HLEM may be a polyalkylene glycol moiety, preferably a PEG. Alternatively, the HLEM is a half-life enhancing polypeptide (HLEP). The HLEP can be a carboxy-terminal peptide (CTP). In one embodiment, the carboxy terminal peptide (CTP) peptide of the present invention comprises the amino acid sequence from amino acid 112 to position 145 of human chorionic gonadotrophin. In another embodiment, the CTP sequence of the present invention comprises the amino acid sequence from amino acid 118 to position 145 of human chorionic gonadotropin, as set forth in SEQ ID NO: 2 (SSSSKAPPPSLPSPSRLPGPSDTPILPQ). In another embodiment, the CTP sequence also commences from any position between positions 112-118 and terminates at position 145 of human chorionic gonadotrophin. In some embodiments, the CTP sequence peptide is 28, 29, 30, 31, 32, 33 or 34 amino acids long and commences at position 112, 113, 114, 115, 116, 117 or 118 of the CTP amino acid sequence.

Alternatively, the HLEP is an FcRn binding partner such as albumin or an immunoglobulin without an antigen binding domain (e.g., Fc). The preferred HLEP of the present invention is albumin.

As used herein, “albumin” refers collectively to albumin polypeptide or amino acid sequence, or an albumin fragment or variant having one or more functional activities (e.g., biological activities) of albumin. In particular, “albumin” refers to human albumin or fragments thereof, especially the mature form of human albumin. For example, albumin can have a sequence or variant thereof, as described in US2008260755A1, which is herein incorporated by reference in its entirety. The albumin portion of the half-life extended FVIIa protein may comprise the full length of the HA sequence, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA.

The terms, human serum albumin (HSA) and human albumin (HA) are used interchangeably herein. The terms, “albumin” and “serum albumin” are broader, and encompass human serum albumin (and fragments and variants thereof) as well as albumin from other species (and fragments and variants thereof). Instead of albumin also other albumin-like proteins, like without limitation human alpha-fetoprotein (as described in WO 2005/024044) as well as their functional fragments or variants may be used.

The albumin portion of the half-life extended FVIIa proteins of the invention may be a variant of normal HA, either natural or artificial. The therapeutic polypeptide portion of the half-life extended FVIIa proteins of the invention may also be variants of the corresponding therapeutic polypeptides as described herein. The term “variants” includes insertions, deletions, and substitutions, either conservative or non-conservative, either natural or artificial, where such changes do not substantially alter the active site, or active domain that confers the therapeutic activities of the therapeutic polypeptides, as described in US2008260755A1, which is herein incorporate by reference in its entirety.

IgG and IgG-fragments may also be used as HLEPs, as long as the HLEP fragments provide a half-life extension of at least 25% as compared to the non-fused coagulation factor. The therapeutic polypeptide portion may be connected to the IgG or the IgG fragments via a linker, preferably a non-cleavable linker.

“Coagulation-related assays” in the sense of the invention is any assay which determines enzymatic or cofactor activities that are of relevance in the coagulation process or that is able to determine that either the intrinsic or the extrinsic coagulation cascade has been activated. The “coagulation-related” assay thus may be direct coagulation assays like aPTT, PT, or the thrombin generation assays. However, other assays like, e.g., chromogenic assays applied for specific coagulation factors are also included. Examples for such assays or corresponding reagents are Pathromtin® SL (aPTT assay, Dade Behring) or Thromborel® S (Prothrombin time assay, Dade Behring) with corresponding coagulation factor deficient plasma (Dade Behring), Thrombin generation assay kits (Technoclone, Thrombinoscope) using e.g. coagulation factor deficient plasma, chromogenic assays like Biophen Factor IX (Hyphen BioMed), Staclot® FVIIa-rTF (Roche Diagnostics GmbH), Coatest® Factor VIII:C/4 (Chromogenix), or others.

Although it is desirable to have a high in vivo recovery and a long half-life for a non-activated coagulation factor, it is advantageous to limit the half-life of a coagulation factor after its activation or the activation of its co-factor in order to avoid a prothrombotic risk. Therefore, after the coagulation process has been initiated, the half-life of the active coagulation factor should again be reduced. This can either be achieved by enhancing inactivation in a coagulation-related mode or by elimination of the coagulation factor.

Inactivation according to the present invention means the decrease of activity of the therapeutic polypeptide which can be caused, for example, by a complex formation of a coagulation factor and an inhibitor of the corresponding coagulation factor or by further proteolytic cleavage as known, e.g., in the case of FVIII and FV.

The inactivation rate of an activated therapeutic half-life extended FVIIa protein is defined as the rate the activity is declining, e.g., by reaction with inhibitors or by proteolytic inactivation. The inactivation rate may be measured by following the molar specific activity of the activated coagulation factor over time in the presence of physiologic amounts of inhibitors of this coagulation factor.

Alternatively, the inactivation rate may be determined after administration of the activated product to an animal followed by testing of plasma samples at an appropriate time frame using activity and antigen assays.

The elimination rate of an activated therapeutic half-life extended FVIIa protein is defined as the rate the polypeptide is eliminated from the circulation of humans or animals. The elimination rate may be determined by measuring the pharmacokinetics of the activated, therapeutic half-life extended FVIIa protein after intravenous administration. Using an antigen assay, the elimination by direct removal from the circulation can be determined. Using an activity assay in addition, a specific removal

As used herein, “acquired hemophilia” refers to a type of hemophilia that develops usually in adulthood from the production of autoantibodies that inhibit the activity or function of FVIII or FIX or accelerate their clearance, in subjects that do not suffer from a previous deficiency of FVIII or FIX, resulting in acquired hemophilia A or B, respectively.

Non-limiting examples of surgical procedures in which half-life extended FVII can be used as therapy to reduce perioperative bleeding include, but are not limited to, cardiac valve surgery (Al Douri et al., 2000. Blood Coag Fibrinol. 11:S121-S127), aortic valve replacement (Kastrup et al., 2002. Ann Thorac Surg. 74:910-912), resection of recurrent hemangiopericytoma (Gerlach et al., 2002. J Neurosurg. 96:946-948), cancer surgery (Sajdak et al., 2002. Eur J Gynaecol Oncol. 23:325-326), and surgery on duodenal ulcers (Vlot et al., 2000. Am J Med. 108:421-423). Subjects undergoing surgery can be given an intravenous bolus of a therapeutic amount of FVII in the early operative phase to reduce perioperative blood loss by enhancing coagulation at the site of surgery. Half-life extended FVIIa polypeptides can be administered to patients with normal coagulation undergoing other types of surgery to effect rapid hemostasis and prevent blood loss. Treatment with a half-life extended FVIIa protein can promote hemostasis at the site of surgery and reduce or prevent blood loss, thereby reducing or abolishing the need for transfusion.

“Trauma” is defined as an injury to living tissue by an extrinsic agent. Trauma is classified as either blunt trauma (resulting in internal compression, organ damage and internal hemorrhage) or penetrative trauma (a consequence of an agent penetrating the body and destroying tissue, vessel and organs, resulting in external hemorrhaging). Trauma can be caused by several events including, but not limited to, vehicle accidents (causing blunt and/or penetrative trauma), gun-shot wounds (causing penetrative trauma), stabbing wounds (causing penetrative trauma), machinery accidents (causing penetrative and/or blunt trauma), and falls from significant heights (causing penetrative and/or blunt trauma). Treatment by administration of therapeutic amounts of FVIIa can promote coagulation and reduce blood loss in trauma patients. For example, a patient with a gun-shot injury presenting with massive blood loss, in addition to surgical intervention, be administered FVIIa to control coagulopathy bleeding. Coagulant therapy with FVIIa can effectively reduce blood loss and hemorrhage in patients with blunt and penetrating trauma (Rizoli et al., 2006. Crit Care. 10:R178).

### Pharmaceutical Compositions and Modes of Administration

The half-life extended FVIIa proteins of the invention can be incorporated into pharmaceutical compositions suitable for administration. Such compositions typically comprise the protein and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical); transmucosal, and rectal administration. Solutions or suspensions used for parenteral, intradermal, or subcutaneous, application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases; such as hydrochloric acid of sodium hydroxide. The parenteral preparation can be enclosed in ampoules, disposable syringes or multiple dose vials made of glass of plastic. Administration as an intravenous injection is the preferred route of administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringe ability exists. It must be stable under the conditions of manufacture and storage and must be preserved against the contaminating action of microorganisms such as bacteria and fungi. The carrier can be a solvent or dispersion medium containing, for examples, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., rVIIa-albumin/rVIIa-FP) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

It is especially advantageous to formulate pharmaceutical compositions, such as compositions for injection, in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved.

The pharmaceutical compositions can be included in a container, pack, or dispenser together with instructions for administration.

Having now described the present invention in detail, the same will be more clearly understood by reference to the following examples, which are included herewith for purposes of illustration only and are not intended to be limiting of the invention. All patents and publications referred to herein are expressly incorporated by reference.

### EXAMPLES

The Examples below were carried out with the half-life extended Factor VIIa protein, comprising recombinant FVIIa (NovoSeven®) and albumin, known as “rVIIa-FP” or “CSL689”. This protein (rVIIa-FP/CSL689) has the amino acid sequence of SEQ ID NO: 1.

### Example 1

### A Single Dose, Placebo-Controlled, Dose-Escalation Safety and PK Study of Half-Life Extended Factor VIIa Protein in 40 Healthy Male Subjects Anticoagulated with Warfarin

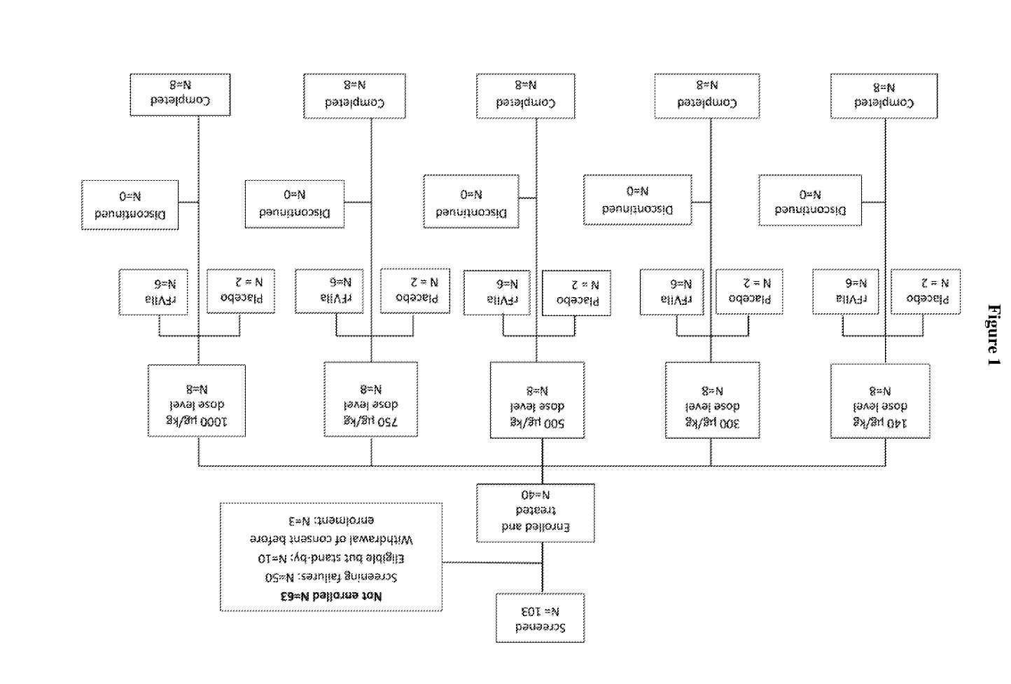

A total of 103 healthy male volunteers aged between 18 and 35 years were screened to enroll 40 subjects in this study. The most relevant reason for screening failure were deviations from the defined in/exclusion criteria within the lab safety parameters, particularly elevated homocystein, decreased protein S and elevated LFTs or failure to reach a stable INR during the warfarin run in phase. Flow of study subjects through the study is shown in FIG. 1. Subject characteristics are depicted in Table 2.

TABLE 2
Characteristics of subjects in the trial

| Characteristic | | rVIIa-FP | | | | | |
|---|---|---|---|---|---|---|---|
| [Unit] | Placebo | 140 μg/kg | 300 μg/kg | 500 μg/kg | 750 μg/kg | 1000 μg/kg | Overall |
| Statistic | (N = 10) | (N = 6) | (N = 6) | (N = 6) | (N = 6) | (N = 6) | (N = 40) |
| Age [years] | | | | | | | |
| Median | 25.5 | 31.0 | 30.5 | 27.5 | 33.0 | 29.0 | 30.0 |
| (min; max) | (21; 33) | (22; 35) | (24; 35) | (23; 35) | (27; 35) | (24; 35) | (21; 35) |
| Height [cm] | | | | | | | |
| Median | 182.0 | 186.0 | 184.0 | 176.0 | 186.5 | 182.5 | 183.0 |
| (min; max) | (173; 190) | (173; 189) | (170; 192) | (170; 188) | (170; 194) | (173; 186) | (170; 194) |
| Weight [kg] | | | | | | | |
| Median | 78.75 | 80.65 | 89.65 | 79.85 | 96.65 | 82.90 | 82.55 |
| (min; max) | (71.1; 88.4) | (65.2; 90.9) | (65.6; 100.2) | (62.9; 97.9) | (69.5; 99.9) | (65.2; 95.5) | (62.0; 100.2) |
| BMI [kg/m²] | | | | | | | |
| Median | 24.10 | 24.25 | 24.55 | 24.70 | 26.45 | 25.30 | 24.80 |
| (min; max) | (21.3; 25.9) | (20.6; 26.3) | (22.6; 29.6) | (21.5; 27.7) | (24.0; 28.4) | (19.3; 28.8) | (19.3; 29.6) |

N = total number of subjects.
Min = minimum.
Max = maximum.
BMI = body mass index.

### Evaluation of Safety (Primary Endpoint):

The local tolerance of rVIIa-FP was good in all subjects. A total of 34 AEs and no SAEs were observed. Three AEs (headache, infusion site haematoma and flatulence) occurred in the placebo group. Thirty-one AEs occurred in the rVIIa-FP groups, with no specific accumulation at any dose level. All AEs except 4 were mild; Four AEs were reported as moderate (nasopharyngitis [2×], ligament sprain and occurrence of several angiolipomas at the forearms). One AE (pain at the infusion site after injection of 1000 μg/kg) was judged to be related to rVIIa-FP. No subject tested positive for anti-drug antibodies or inhibitors before, at day 8, or on day 28 after study drug administration. One subject at the 500 μg/kg dose of rVIIa-FP reported a sudden appearance of several subcutaneous nodules on the left forearm and the upper right arm. This subject had multiple pre-existing lipomas on both forearms. The newly reported nodules were manually movable subcutaneous nodules and showed no signs of inflammation. A biopsy was performed, and histology showed nodular tumors of lipocytes crossed by capillary proliferations with individual microthrombi. These results are consistent with an angiolipoma (teleangiectatic lipoma), which is considered normal for a male of that age. In addition, immunohistochemical investigation showed no proliferative activity (Ki-67) in the lipocytes. There was nuclear expression in some endothelial cells of the capillary proliferations within the angiolipoma (<5%). This result is not consistent with rapid growth resulting in a sudden appearance as reported by the subject. No relationship to administration of rVIIa-FP could be established.

All AEs resolved without sequelae with the exception of angiolipoma that was reduced in size but still detectable at the end of study.

The safety analysis had special focus on thromboembolic complications. No thromboembolic events were observed during the study. One subject (receiving rVIIa-FP at the 1000 μg/kg dose) experienced pain at the infusion site but this was not related to a thrombophlebitic or thrombotic reaction.

Evaluation of the pharmacokinetic profile of rVIIa-FP (secondary endpoint): Prior to infusion of rVIIa-FP, FVIIa activity was below the level of quantitation of the assay (7.8 mU/mL) in all subjects except one who had a baseline FVIIa activity of 8.6 mU/mL. The PK parameters are summarized in Table 3.

TABLE 3
Descriptive Summary of PK Parameters - Baseline Corrected (PK population)

| | | 140 μg/kg | 300 μg/kg | 500 μg/kg | 750 μg/kg | 1000 μg/kg |
|---|---|---|---|---|---|---|
| | Statistic | (N = 6) | (N = 6) | (N = 6) | (N = 6) | (N = 6) |
| IR | Mean | 67.97 | 71.58 | 56.35 | 60.08 | 65.39 |
| [(mU/mL)/(μg/kg)] | SD | 3.867 | 8.718 | 5.271 | 5.030 | 13.913 |
| Cmax [mU/mL] | Mean | 9240.0 | 20873.3 | 27451.9 | 43760.0 | 63520.0 |
| | SD | 515.36 | 2552.79 | 2544.67 | 3668.59 | 13515.05 |
| t½ [h] | Mean | 6.055* | 13.471* | 12.267* | 7.549 | 8.512 |
| | SD | 0.9609 | 12.4203 | 8.5886 | 0.7392 | 0.9640 |
| AUC₀₋ₜ [h*mU/mL] | Mean | 49546.4 | 151200.7 | 234641.0 | 371649.2 | 616637.5 |
| | SD | 7108.88 | 16601.70 | 35680.36 | 71741.77 | 125521.98 |
| AUC0-∞[h*mU/mL] | Mean | 50760.5* | 155913.6* | 237998.6* | 371774.4 | 616805.8 |
| | SD | 7359.02 | 13830.68 | 38761.94 | 71774.41 | 125547.08 |
| CL [mL/h/kg] | Mean | 12.74* | 8.81* | 9.78* | 9.45 | 7.62 |
| | SD | 1.760 | 0.774 | 1.791 | 1.769 | 1.499 |
| Vss [mL/kg] | Mean | 85.20* | 81.70* | 97.09* | 97.97 | 84.57 |
| | SD | 9.338 | 12.395 | 13.420 | 13.263 | 12.760 |
| MRT_{0-∞} [h] | Mean | 6.771* | 9.264* | 10.189* | 10.556 | 11.187 |
| | SD | 1.0487 | 1.0242 | 2.4951 | 1.6909 | 0.7229 |

*N = 5

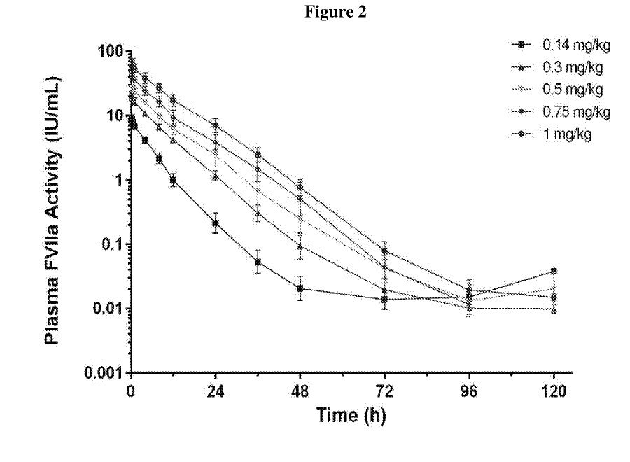

Following single IV infusions of 140 μg/kg to 1000 μg/kg, FVIIa activity peaked near the end of infusion (FIG. 2). The median tₘₐₓ was 0.267 to 0.500 hours across dose levels, which corresponded to the end of infusion for approximately half of the subjects. FVIIa baseline-corrected mean Cₘₐₓ (SD) values increased in a dose-proportional manner, with an approximately 7-fold increase in Cₘₐₓ over the 7-fold increase in dose (9240 (515) mU/mL for the 140 μg/kg dose to 63520 (13515) mU/mL for the 1000 μg/kg dose). Therefore, the IR was fairly consistent across dose levels and ranged from 56.35 [(mU/mL)/(μg/kg)] at the 500 μg/kg dose level to 71.58 [(mU/mL)/(μg/kg)] at the 300 μg/kg dose level. This is consistent with the reported behavior of rFVIIa (Lindley C M, et al., 1994. Clinical pharmacology and therapeutics. 55(6):638-48).

The baseline-corrected mean FVIIa AUC₀₋ₜ increased in a slightly more than dose-proportional manner across the dose range, from 49546 (7109) h*mU/mL for the 140 μg/kg dose to 616638 (125522) h*mU/mL for the 1000 μg/kg dose.

The individual values of mean half-life were documented in the range of 6.1 to 13.5 hours. The CV % for half-life was high for the 300 μg/kg and 500 μg/kg dose groups (92% and 70%, respectively). This was due to one subject in each cohort with a long t_{1/2} compared to the other subjects in those dose groups. For 1 subject in each of the lower 3 dose groups, the elimination rate constant and related PK parameters could not be estimated due to variation of the last several time points around baseline. Across the dose range, the median t_{1/2} was quite consistent ranging from 6.1 hours to 9.7 hours. At the highest dose (1000 μg/kg) the median t_{1/2} was 8.5 hours.

Assessment of dose proportionality using the power-law model showed that baseline-corrected and uncorrected Cmax increased in a dose-proportional manner across the 140 μg/kg to 1000 μg/kg dose levels; the slope estimate was 0.9351 with a corresponding 95% confidence interval of (0.8606-1.0095). In contrast, AUC₀₋ₜ increased in a more than dose-proportional manner, since the baseline corrected slope estimate was 1.2205 with a corresponding confidence interval of (1.1250-1.3160). Therefore, the increase in mean AUC₀₋ₜ was 22% higher than expected compared with the corresponding increase in dose.

CL was consistent across dose levels, ranging from 7.62 to 12.74 [mL/h/kg]. Volume of distribution (Vss) was also consistent, ranging from 81.70 to 97.97 [mL/kg].

### Example 2

### Prophylactic Administration of Half-Life Extended Factor VIIa Protein

Subjects participating in the CSL689 on demand study will be offered participation in the prophylaxis trial. The prophylaxis trial will compare the annualized bleeding rate under prophylaxis with the annualized bleeding rate documented in the CSL689_2001 study. The prophylaxis trials are summarized in the below Table 4.

TABLE 4
Clinical Trials

| Study identifier | Type of study | Study population | Dosage, regimen | Primary endpoint(s) |
|---|---|---|---|---|
| Completed | | | | |
| CSL689_1001 | Phase I | Healthy male | CSL689 or placebo | Frequency of related AEs to |
| (FIH) | Safety and PK | subjects | IV administration | rVIIa-FP over the course of |
| | | 18 to 35 yrs | 140, 300, 500, 750, | the study. |
| | | | 1000 μg/kg | Occurrence of inhibitors |
| | | | | against FVII. |
| | | | | Occurrence of antibodies |
| | | | | against rVIIa-FP. |
| Planned | | | | |
| CSL689_2001 | Phase II/III | Male subjects with | CSL689 | PK Module: |
| (on-demand + | Safety, Efficacy | haemophilia A and | IV administration | PK of CSL689 and |
| surgical | and PK | B with inhibitors | 0.75 and 1.5 mg/kg | NovoSeven rFVIIa, based |
| interventions) | | Blocks A and | Comparator in PK | on plasma rFVIIa activity |
| | | B: ≧12 to ≦65 yrs | Module for Block | with and without baseline |
| | | Block | A: rFVIIa | correction. PK endpoints: |
| | | C: <12 yrs (Cohort | (NovoSeven ®) | Area under concentration |
| | | C1) ≧12 to ≦65 yrs | 90 and 270 μg/kg | versus time curve from time |
| | | (Cohort C2) | | zero to last sample (AUC₀₋ₜ). |
| | | | | AUC extrapolated to |
| | | | | infinity (AUC_{0-∞}). |
| | | | | Maximum observed plasma |
| | | | | concentration (Cₘₐₓ). |
| | | | | Time corresponding to |
| | | | | occurrence of Cₘₐₓ (tₘₐₓ). |
| | | | | Incremental recovery (IR). |
| | | | | Terminal elimination half- |
| | | | | life (t_{1/2}). |
| | | | | Clearance (CL). |
| | | | | Dose-evaluation Module: |
| | | | | Percentage of bleeding |
| | | | | events successfully treated |
| | | | | with the first injection of |
| | | | | CSL689 at each dose level. |
| | | | | Repeated-dose Module: |
| | | | | Percentage of bleeding |
| | | | | events successfully treated |
| | | | | with the first injection of |
| | | | | CSL689 at the population- |
| | | | | based best dose in Blocks |
| | | | | A, B, and C. |
| CSL689_3001 | Phase II/III | Male subjects with | CSL689 | Reduction of break-through |
| (prophylaxis) | Safety, Efficacy | haemophilia A and | IV administration | bleeding events on |
| | and PK | B with inhibitors | Prophylaxis | prophylaxis schedule(s) |
| | | completing study | regimen depending | compared to annualised |
| | | CSL689_2001 | on dose-evaluation | bleeding rate under on- |
| | | ≦65 yrs | data from preceding | demand treatment in Study |
| | | | Study CSL689_2001 | CSL689_2001 |

Due to the properties of CSL689 it is anticipated that a prophylactic dose of 1.5 mg/kg can be effective and that the dosing interval can be greater than previously achieved with rVIIa, e.g., once every two days (i.e. once every other day). Potentially, lower doses may also be effective in such a prophylactic dosing regimen, such as 1 mg/kg or 0.75 mg/kg.

### Example 3

### The Half-Life Extended FVIIa Protein More Effectively Controls Bleeding Compared to rFVIIa After Standardized Kidney Injury (A Model for Bleeding in Surgery and Trauma) in a Rabbit Model of Dilutional Coagulopathy

### Material & Methods

### Animals

Female CHB rabbits 3-4 months old weighing 2.0-4.0 kg (Bauer, Neuental, Germany) were housed one per cage in wire-steel cages at 21-23° C. and 50% relative humidity under a 12 h/12 h light-darkness cycle. The animals were provided tap water ad libitum and fed rabbit pellets (Deukanin®, Deutsche Tiernahrung Cremer GmbH & Co. KG, Dusseldorf, Germany). All rabbits received care in compliance with the European Convention on Animal Care, and the study was approved by the organizational Ethics Committee.

### Hemodilution

All treatments were conducted in anesthesized animals, Anesthesia was induced by a combination of ketamine and xylazine and maintained via inhalative isoflurane anesthesia. The animals were then intubated and placed on a ventilator (Heyer Access, Heyer Medical AG, Bad Ems, Germany).

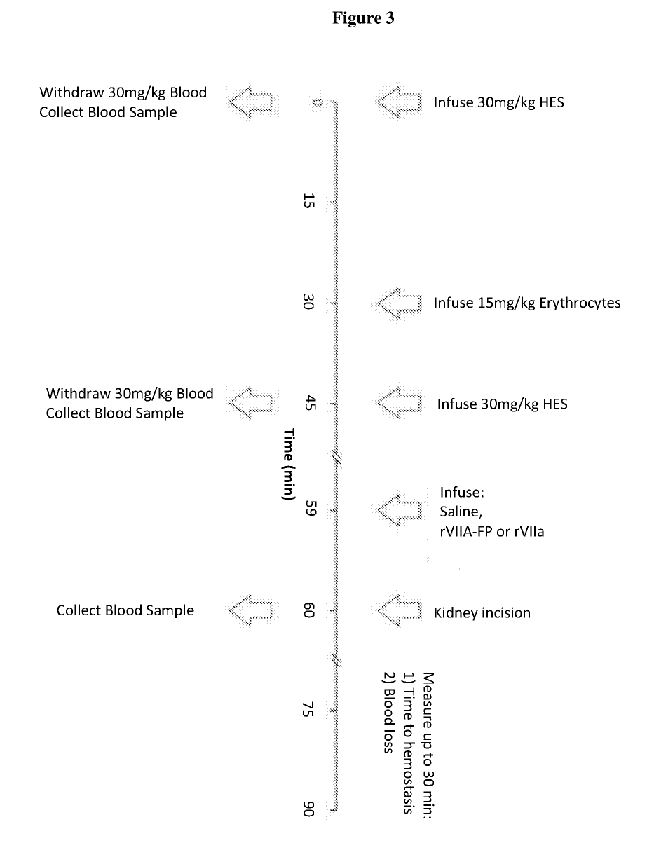

Animals were subjected to hemodilution in phases by withdrawal of 30 mL·kg⁻¹ blood and infusion of 30 mL·kg⁻¹ hydroxyethyl starch (HES) 200/0.5 (Infukoll 6%, Schwarz Pharma AG, Mannheim, Germany) prewarmed to 37° C. from the carotid artery (FIG. 3). That procedure was repeated at 45 min. At 30 min, during the interval between the two cycles of blood withdrawal and HES infusion, the animals received 15 mL·kg⁻¹ salvaged erythrocytes, prepared from withdrawn whole rabbit blood by centrifugation for 10 min at 800×g, washing in normal saline and resuspension in Ringer's lactate, administered into the external jugular vein.

### Kidney Injury

At 60 min after commencement of hemodilution, a standardized renal injury was inflicted in the form of a 15 mm long and 5 mm deep scalpel incision at the lateral kidney pole (FIG. 3).

### Treatment

Animals were randomly allocated receive i.v. administrations of isotonic saline, rVIIa-FP (CSL Behring GmbH, Marburg, Germany) at doses of 0.75 mg/kg or 1.5 mg/kg, or rFVIIa (NovoSeven®, Novo Nordisk A/S, Bagsværd, Denmark) at doses of 90 μg/kg or 180 μg/kg immediately prior to kidney incision injury (FIG. 3). Experimental groups consisted of 5 rabbits each.

Dose levels were selected to achieve comparable FVIIa activity levels of rVIIa-FP and rFVIIa based on the Staclot® assay. Consequently, 0.75 mg/kg rVIIa-FP correlates to 90 μg/kg rFVIIa based on FVIIa activity, whereas 1.5 mg/kg rVIIa-FP correlates to 180 μg/kg rFVIIa based on FVIIa activity.

### Endpoints

The primary study endpoints were time to hemostasis and blood loss as observed up to 30 min following a standardized kidney incision injury (FIG. 3). Time to hemostasis was defined as the interval from the kidney incision until cessation of observable bleeding or oozing. Blood loss was the volume of blood collected from the incision site by suction. The 30 min observation period for blood loss and time to hemostasis began immediately after the incision.

### Results

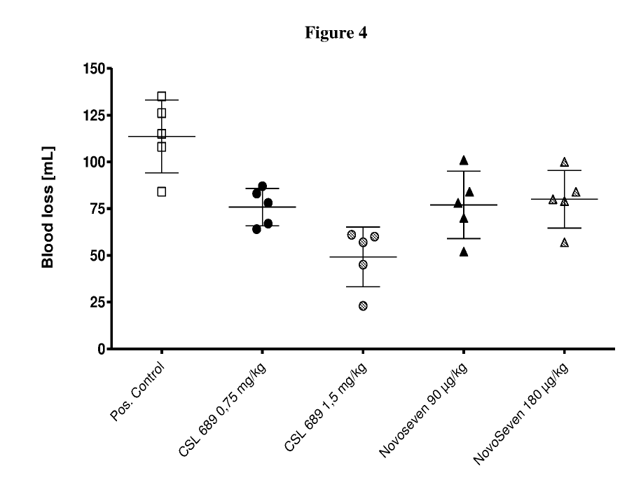

As shown in FIG. 4, NovoSeven® at doses of 90 and 180 μg/kg was able to reduce total blood loss following standardized kidney injury from an average of 115 mL (untreated; positive control) to 77 and 80 mL, respectively, therefore not exhibiting any apparent dose-response. In contrast, CSL689 (rVIIa-FP) was equally effective at a dose 0.75 mg/kg (average blood loss of 76 mL) and even more effective at the higher dose of 1.5 mg/kg which lead to a reduction in blood loss to only 49 mL. Therefore, CSL689 showed a clear dose-response effect regarding bleeding reversal in hemodiluted rabbits and was more effective compared to comparable doses of rFVIIa (NovoSeven®).

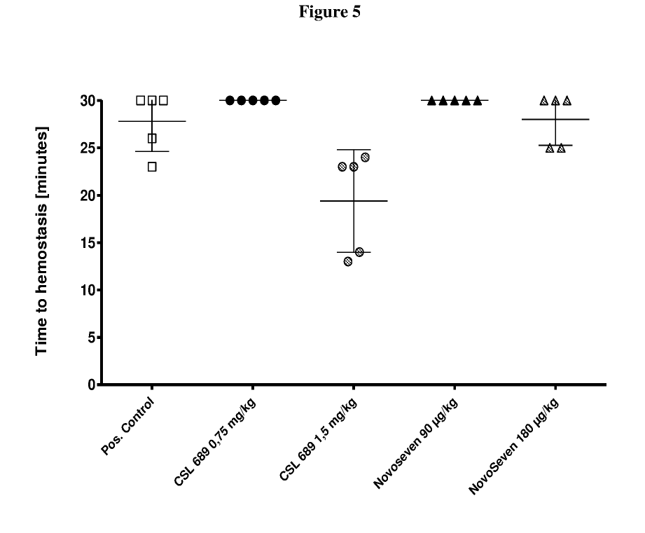

In addition, only rVIIa-FP was able to markedly reduce the time required to achieve complete hemostasis following standardized kidney injury in these animals (FIG. 5). In contrast, no or only small effects were observed following treatment with rFVIIa (NovoSeven®), confirming improved efficacy of CSL689 regarding bleeding reversal following acute trauma.

### Example 4

### Efficacy of the Half-Life Extended FVIIa Protein in a Monkey Model of Acquired Hemophilia

### Introduction/Objective

The study was conducted to assess the efficacy of the recombinant fusion protein linking activated Factor VII with human albumin, i.e. rVIIa-FP (CSL689 (SEQ ID NO: 1)), in cynomolgus monkeys with circulating anti-Factor VIII antibodies which mimic the clinical conditions of patients with acquired hemophilia A. In addition, a direct comparison between rVIIa-FP and recombinant activated Factor VII (rFVIIa; NovoSeven®), regarding the dosing frequency required for effective bleeding prophylaxis was included.

### Material and Methods

### Animal Management

All in-life experimental procedures were approved by the local animal welfare authorities.

Female cynomolgus monkeys (Macaca fascicularis; purpose bred), at least 2 years old at the start of treatment, were acclimatized to housing conditions in the primate building for up to 4 weeks before the start of treatment. The health status of the animals was reviewed by a veterinary officer and confirmed acceptable prior to the start of treatment. Animals (n=4 per group) were allocated to treatment groups based on a pseudo-random bodyweight and baseline plasma Factor VIII (FVIII) activity stratification procedure yielding groups with approximately equal mean bodyweight and FVIII activity baseline levels. The animals were given unique new identity numbers. Animals were housed in pairs in a temperature and humidity controlled area with 12 hours light, 12 hours dark lighting. Each animal was offered a restricted diet (200 g of a standard dry diet and biscuit and fruit supplements) and had free access to water.

### Treatments

Cynomolgus monkeys were immunized by subcutaneous administration of a mixture of FVIII and adjuvant on Day 1. Upon development of the acquired hemophilia phenotype as indicated by FVIII activity levels in plasma, animals received an intravenous bolus dose of rVIIa-FP (1.5 mg/kg) or rFVIIa (90 μg/kg) into the cephalic or saphenous veins starting with a dosing interval of 8 and 3 hours, respectively. Once satisfactory bleeding control was achieved based on clinical observations and hematology over at least 48 hours or 2 subsequent doses, whatever is longer, the dosing interval was increased. The following dosing intervals were applied:
rVIIa-FP: 8, 12, 16, 32 hours
rFVIIa: 3, 6, 12, 24 hours

### Endpoints Evaluated

Serial observations included clinical observations, mortality, body weight (at least once weekly and before necropsy), food consumption, hematology including determination of FVIII activity levels in plasma, hematocrit, hemoglobin concentration, prothrombin time (PT and activated partial thromboplastin time (aPTT), blood chemistry, bone marrow smears, anti-FVIII antibodies; anti-FVII antibodies; neutralizing FVIII antibodies; FVIIa activity (Staclot® assay); macroscopic pathology, histopathology.

### Results

Cynomolgus monkeys were immunized by subcutaneous administration of a mixture between Factor VIII and adjuvant. Subsequently, animals produced anti-Factor VIII antibodies which were cross-reactive to endogenous monkey Factor VIII leading to a bleeding phenotype closely reflecting the clinical bleeding phenotype observed in patients with acquired hemophilia A based on clinical observations, hematology and macroscopic as well as microscopic evaluations in the absence of treatment. Results confirmed that FVIII activity levels in plasma are the most sensitive endpoints predictive of the successful induction of this acquired hemophilia A phenotype correlating well with the appearance of anti-FVIII antibodies cross-reactive to monkey FVIII. Therefore, prophylactic rVIIa-FP or rFVIIa treatment was initiated once FVIII activity levels have dropped below 10% (0.1 IU/m).

Overall, the results confirmed that the maximum dosing interval effectively protecting all animals from developing any bleeding symptoms in the presence of inhibitors for rVIIa-FP treatment at a dose of 1.5 mg/kg was 8 hours. In comparison, the maximum dosing interval for NovoSeven® at a dose of 90 μg/kg required to effectively protect all animals treated was 3 hours.

1-69. (canceled)

70. A method of preventing bleeding, comprising administering a half-life extended Factor VIIa (FVIIa) protein to a subject,
wherein the half-life extended FVIIa protein comprises
(a) a FVIIa portion, and
(b) a half-life enhancing moiety (HLEM);
and wherein the half-life extended FVIIa protein is administered at a dose
(a) resulting in a Cmax of at least about 30 IU per ml of blood;
(b) comprising about 200 μg/kg to about 800 μg/kg of the FVIIa portion; or
(c) comprising about 1750 IU/kg to about 8000 IU/kg of the FVIIa portion.

71. The method of claim 70, wherein the Cmax or the activity (IU) is determined with a Staclot® assay.

72. The method of claim 70, wherein the half-life extended FVIIa protein is administered at a dose resulting in a Cmax of about 30 IU per ml of blood to about 160 IU per ml of blood, about 35 IU per ml of blood to about 130 IU per ml of blood, about 40 IU per ml of blood to about 105 IU per ml of blood, about 46 IU per ml of blood to about 92 IU per ml of blood, about 30 IU per ml of blood to about 70 IU per ml of blood, about 36 IU per ml of blood to about 56 IU per ml of blood, about 41 IU per ml of blood to about 51 IU per ml of blood, about 70 IU per ml of blood to about 110 IU per ml of blood, about 80 IU per ml of blood to about 105 IU per ml of blood, about 82 IU per ml of blood to about 102 IU per ml of blood, or about 87 IU per ml of blood to about 97 IU per ml of blood.

73. The method of claim 70, wherein the half-life extended FVIIa protein is administered at a dosing interval of once about every 1 to 5 days, once about every 2 to 4 days, once about every 2 to 3 days, once about every other day, or at least once every day.

74. The method of claim 70, wherein the dose comprises about 300 μg/kg to about 700 μg/kg, about 320 μg/kg to about 650 μg/kg, about 310 μg/kg to about 330 μg/kg, or about 630 μg/kg to about 650 μg/kg of the FVIIa portion.

75. The method of claim 70, wherein the dose comprises about 1800 IU/kg to about 7000 IU/kg, about 2500 IU/kg to about 6500 IU/kg, about 3200 IU/kg to about 5800 IU/kg, about 2800 IU/kg to about 3200 IU/kg, or about 5800 IU/kg to about 6200 IU/kg of the FVIIa portion.

76. A method of treating a bleeding episode, comprising administering a half-life extended Factor VIIa (FVIIa) protein to a subject,
wherein the half-life extended FVIIa protein comprises
(a) a FVIIa portion, and
(b) a half-life enhancing moiety (HLEM);
and wherein the half-life extended FVIIa protein is administered at a dose
(a) resulting in a Cmax of at least about 30 IU per ml of blood;
(b) comprising about 200 μg/kg to about 800 μg/kg of the FVIIa portion; or
(c) comprising about 1750 IU/kg to about 8000 IU/kg of the FVIIa portion.

77. The method of claim 76, wherein the Cmax or the activity (IU) is determined with a Staclot® assay.

78. The method of claim 76, wherein the first administration leads to termination of bleeding in 30% of patients.

79. The method of claim 76, further comprising administering a second dose of the half-life extended FVIIa protein to the subject, wherein the second dose is identical to the first dose, and wherein the second dose is administered at a dosing interval of about 5 to 10 hours after the first dose or the second dose is administered at a dosing interval of 6 to 8 hours after the first dose.

80. The method of claim 79, wherein the second dose leads to termination of bleeding in 60% of patients.

81. The method of claim 76, wherein the bleeding episode is a result of trauma.

82. The method of claim 81, wherein administration results in is a reduction in transfusion requirements by at least 20% within 24 hours after administration.

83. A method of preventing a bleeding episode during or after surgery, comprising administering a half-life extended Factor VIIa (FVIIa) protein to a subject,
wherein the half-life extended FVIIa protein comprises
(a) a FVIIa portion, and
(b) a half-life enhancing moiety (HLEM);
and wherein the half-life extended FVIIa protein is administered at a dose
(a) resulting in a Cmax of at least about 30 IU per ml of blood;
(b) comprising about 200 μg/kg to about 800 μg/kg of the FVIIa portion; or
(c) comprising about 1750 IU/kg to about 8000 IU/kg of the FVIIa portion;
and wherein the half-life extended FVIIa protein is administered before, during, and/or after surgery.

84. The method of claim 83, wherein the Cmax or the activity (IU) is determined with a Staclot® assay.

85. The method of claim 83, further comprising administering a second dose of the half-life extended FVIIa protein to the subject, wherein the second dose is identical to the first dose, and wherein the second dose is administered at a dosing interval of about 5 to 10 hours after the first dose or the second dose is administered at a dosing interval of about 6 to 8 hours after the first dose.

86. The method of claim 73, wherein a hemostatic potential is maintained at a trough of at least 1% for the entire dosing interval.

87. The method of claim 70, wherein the half-life of the half-life extended FVIIa protein is greater than about 5 hours.

88. The method of claim 70, wherein the half-life enhancing moiety (HLEM) is a polyalkylene glycol moiety.

89. The method of claim 88, wherein the polyalkylene glycol moiety is polyethylene glycol (PEG).

90. The method of claim 70, wherein the half-life enhancing moiety (HLEM) is a half-life enhancing polypeptide (HLEP).

91. The method of claim 90, wherein the HLEP is a carboxy-terminal peptide (CTP) or a neonatal Fc receptor (FcRn) binding partner.

92. The method of claim 91, wherein the FcRn binding partner is albumin or an immunoglobulin without an antigen binding domain.

93. The method of claim 90, wherein the HLEP is albumin.

94. The method of claim 93, wherein the dose of the half-life extended FVIIa protein is at least about 500 μg/kg, about 500 μg/kg to about 2500 μg/kg, about 750 μg/kg to about 2000 μg/kg, about 1000 μg/kg to about 1500 μg/kg, about 1200 μg/kg to about 1500 μg/kg, about 740 μg/kg to about 760 μg/kg, or about 1490 μg/kg to about 1510 μg/kg.

95. A method of preventing bleeding, comprising administering a half-life extended Factor VIIa (FVIIa) protein to a subject,
wherein the half-life extended FVIIa protein comprises
(a) a FVIIa portion, and
(b) a half-life enhancing polypeptide (HLEP);
and wherein the half-life extended FVIIa protein is administered at a dosing interval of once about every 2 to 4 days.

96. The method of claim 95, wherein the HLEP is a carboxy-terminal peptide (CTP) or an FcRn binding partner.

97. The method of claim 96, wherein the FcRn binding partner is albumin or an immunoglobulin without an antigen binding domain.

98. The method of claim 95, wherein the dosing interval is once about every 2 to 3 days or once about every other day.

99. The method of claim 70, wherein the half-life extended FVIIa protein has a sequence as set forth in SEQ ID NO:1.

100. The method of claim 70, wherein the method involves a prophylactic dosing regime.

101. The method of claim 70, wherein the subject suffers from hemophilia A, hemophilia B, or acquired hemophilia, or wherein the subject has inhibitory antibodies against Factor VIII and/or Factor IX, or Congenital Hemophilia with Inhibitors (CHwI).

102. The method of claim 70, wherein the half-life extended FVIIa protein is administered intravenously.


SEQUENCE LISTING (2 sequences)

SEQ ID NO: 1 - protein, 1022 aa, Artificial Sequence
  Fusion protein N-terminal human FVII followed by flexible linker followed by human serum albumin
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPSSGGSGGSGGSGGSGGSGGSGGSGGSGGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 28 aa, Homo sapiens
SSSSKAPPPSLPSPSRLPGPSDTPILPQ